SEQUENCE LISTING (10 sequences)

SEQ ID NO: 1 - RNA, 193 nt, Artificial
  Exon 10 of the CFTR transcript
gacuucacuucuaauggugauuaugggagaacuggagccuucagaggguaaaauuaagcacaguggaagaauuucauucuguucucaguuuuccuggauuaugccuggcaccauuaaagaaaauaucaucuuugguguuuccuaugaugaauauagauacagaagcgucaucaaagcaugccaacuagaagag

SEQ ID NO: 2 - DNA, 10 bp, Artificial
  Oligonucleotide
tactaactgt

SEQ ID NO: 3 - DNA, 5 bp, Artificial
  Oligonucleotide
ctaat

SEQ ID NO: 4 - DNA, 19 bp, Artificial
  Oligonucleotide
cctttcttcttttccttcc

SEQ ID NO: 5 - DNA, 10 bp, Artificial
  Oligonucleotide
ttttatttcc

SEQ ID NO: 6 - DNA, 6 bp, Artificial
  Oligonucleotide
gtaagt

SEQ ID NO: 7 - DNA, 9 bp, Artificial
  Oligonucleotide
tccctccag

SEQ ID NO: 8 - RNA, 566 nt, Artificial
  Polynucleotide
uccaauuaucauccuaagcagaaguguauauucuuauuuguaaagauucuauuaacucauuugauucaaaauauuuaaaauacuuccuguuucagguacucugcuaugcacaaaagauacaagggaaaguaaaagagacaggcaagugaauccugagcgugauuugauaaugaccuaauaaugauggguuuuauuuccagacuucacuucuaauggugauuaugggagaacuggagccuucagaggguaaaauuaagcacaguggaagaauuucauucuguucucaguuuuccuggauuaugccuggcaccauuaaagaaaauaucaucuuugguguuuccuaugaugaauauagauacagaagcgucaucaaagcaugccaacuagaagagguaagaaacucucuuucuuuccauggguuggccuugauccauucacaguagcuuacccauagaggaaacauaaauauauguagacuaaccgauugaauauggagccaaauauauaauuuggguagugugaaggguucauaugcauaaucaaaaaguuuucacauaguuucuuac

SEQ ID NO: 9 - RNA, 548 nt, Artificial
  Polynucleotide
uccaauuaucauccuaagcagaaguguauauucuuauuuguaaagauucuauuaacucauuugauucaaaauauuuaaaauacuuccuguuucagguacucugcuaugcacaaaagauacaagggaaaguaaaagagacagauaaugaccuacuaacugugccuuucuucuuuuccuuccagacuucacuucuaauggugauuaugggagaacuggagccuucagaggguaaaauuaagcacaguggaagaauuucauucuguucucaguuuuccuggauuaugccuggcaccauuaaagaaaauaucaucuuugguguuuccuaugaugaauauagauacagaagcgucaucaaagcaugccaacuagaagagguaagaaacucucuuucuuuccauggguuggccuugauccauucacaguagcuuacccauagaggaaacauaaauauauguagacuaaccgauugaauauggagccaaauauauaauuuggguagugugaaggguucauaugcauaaucaaaaaguuuucacauaguuucuuac

SEQ ID NO: 10 - RNA, 548 nt, Artificial
  Polynucleotide
ugccaagugcucacucugugucgagugcuguucuaugugcuuuaacuauauuaauuuauuuaaucuucacagaaauccuacaaaguagauuaccuucauauuauuagguacagauuaaguaauagagacauauucagguagauaaugaccuacuaacugugccuuucuucuuuuccuuccagacuucacuucuaauggugauuaugggagaacuggagccuucagaggguaaaauuaagcacaguggaagaauuucauucuguucucaguuuuccuggauuaugccuggcaccauuaaagaaaauaucaucuuugguguuuccuaugaugaauauagauacagaagcgucaucaaagcaugccaacuagaagagguaagaaacucucuuucuuuccauggguuggccuaaauaaucuuaauaauuuuuggaguauauuuuuaaagaugcauauuuugugguaucuuuuaaaaagauaccacauaucacuuauaugcaugccauauaaauaaccauugaggacguuugucucacuaaugagugaacaaa

(19) United States
(12) Patent Application Publication
(10) Pub. No.: US 2015/0209448 A1
(43) Pub. Date: Jul. 30, 2015

(54) EXON REPLACEMENT WITH STABILIZED ARTIFICIAL RNAS

(71) Applicant: ProQR Therapeutics N.V., Leiden (NL)

(72) Inventors: Daniel Anton de Boer, Putten (NL); Tita Ritsema, Utrecht (NL)

(21) Appl. No.: 14/414,313

(22) PCT Filed: Jul. 12, 2013
(86) PCT No.: PCT/NL2013/050531

§ 371 Date: Jan. 12, 2015

Related U.S. Application Data
(60) Provisional application No. 61/670,659, filed on Jul. 12, 2012.

(51) Int. Cl. A61K 48/00; A61K 45/06; C12N 15/113; C07K 14/705

(52) U.S. Cl. CPC A61K 48/0066; C07K 14/705; A61K 45/06; C12N 15/113; C12N 2320/33

(57) ABSTRACT

The present invention relates to the field of gene therapy, more specifically to the use of stabilized artificial RNA molecules for trans-splicing reactions to replace faulty exons for healthy exons. The present invention further relates to the use of the stabilized RNA molecules for treatment of genetic diseases.

### FIELD OF THE INVENTION

The present invention relates to the field of gene therapy, more specifically to the use of stabilized artificial RNA molecules for trans-splicing reactions to replace faulty exons for healthy exons. The present invention further relates to the use of the stabilized RNA molecules for treatment of genetic diseases.

### BACKGROUND OF THE INVENTION

Splicing is the process in which exons of the pre-mRNA are assembled into mRNA. Generally splicing takes place within one pre-mRNA molecule and called cis-splicing. Sometimes splicing takes place between more than one pre-mRNA molecule, which is called trans-splicing. Trans-splicing was discovered in trypanosomes, but has by now been described in most kingdoms, including in man (Horiuchi T, Aigaki T. Alternative trans-splicing: a novel mode of pre-mRNA processing. Biol Cell. 2006 February; 98(2):135-40).

Trans-splicing is considered a relatively rare event in nature, but has been performed with the relative high efficiency in artificial settings. For this purpose cells were transfected with DNA encoding a trans-splicing molecule which contained besides the trans-splicing exons for example a region which made the trans-splicing molecule capable of base-pairing to the original pre-mRNA. Trans-splicing was described to create healthy mRNA for several genetic diseases such as haemophilia A (Chao H, Mansfield S G, Bartel R C, Hiriyanna S, Mitchell L G, Garcia-Blanco M A, Walsh C E. Phenotype correction of hemophilia A mice by spliceosome-mediated RNA trans-splicing. Nat Med. 2003 August; 9(8):1015-9), spinal muscular atrophy (Coady T H, Baughan T D, Shababi M, Passini M A, Lorson C L. Development of a single vector system that enhances trans-splicing of SMN2 transcripts. PLoS One. 2008; 3(10):e3468), X-linked immunodeficiency (Tahara M, Pergolizzi R G, Kobayashi H, Krause A, Luettich K, Lesser M L, Crystal R G. Trans-splicing repair of CD40 ligand deficiency results in naturally regulated correction of a mouse model of hyper-IgM X-linked immunodeficiency. Nat Med. 2004 August; 10(8):835-41) and cystic fibrosis (Liu X, Luo M, Zhang L N, Yan Z, Zak R, Ding W, Mansfield S G, Mitchell L G, Engelhardt J F. Spliceosome-mediated RNA trans-splicing with recombinant adeno-associated virus partially restores cystic fibrosis transmembrane conductance regulator function to polarized human cystic fibrosis airway epithelial cells. Hum Gene Ther. 2005 September; 16(9):1116-23). Trans-splicing makes use of the cell's endogenous splicing machinery.

In the examples mentioned above there was one trans-splicing event needed. For this single trans-splicing the exons before or after the exon-to-be-repaired need to be encoded on the trans-splicing molecule. The more used approach is 3′ trans-splicing, where the 3′part of the mRNA, including the faulty exon is derived from the sequence provided by the trans-splicing molecule. In case of 5′ trans-splicing the 5′ part of the mRNA is derived from the trans-splicing molecule. Due to the long sequences needed, trans-splicing molecules are generally delivered using viral delivery systems, like those used for gene therapy. In these systems a DNA molecule is delivered to the cell, from which the trans-splicing RNA needs to be transcribed.

Double trans-splicing leads to the replacement of one exon within the mRNA. For this to occur two trans-splicing events are needed, one 3′ of the exon of interest, the other one 5′ of the exon of interest. This phenomenon was described using an artificial system where the target pre-mRNA was derived from a minigene encoded on a plasmid. A host cell was transiently transfected with the target pre-mRNA-encoding a plasmid and the trans-splicing molecule and exon replacement could be detected (Lorain S, Peccate C, Le Hir M, Garcia L. Exon exchange approach to repair Duchenne dystrophin transcripts. PLoS One. 2010 May 28; 5(5):e10894).

The use of stabilized mRNA is described as a tool for the production of therapeutic protein (Kormann M S, Hasenpusch G, Aneja M K, Nica G, Flemmer A W, Herber-Jonat S, Huppmann M, Mays L E, Illenyi M, Schams A, Griese M, Bittmann I, Handgretinger R, Hartl D, Rosenecker J, Rudolph C. Expression of therapeutic proteins after delivery of chemically modified mRNA in mice. Nat Biotechnol. 2011 February; 29(2):154-7). In vitro DNA transcription in the presence of chemically modified nucleotide monomers leads to the synthesis of stabilized mRNA. The modified monomers were 2-thiouridine and 5-methyl-cytidine, which were added up to 25% of total uridine and cytidine respectively. When such a stabilized mRNA for lung surfactant protein B (SPB) was given to mice deficient for SPB, SPB was produced.

### DESCRIPTION OF THE INVENTION

In general terms, exon replacement is a mechanism that enables the exchange of a (faulty) piece of RNA for another (healthy) one. The actual exchange takes place during splicing when an exon from an artificial piece of RNA is included in the mRNA instead of the naturally occurring faulty exon. The result is that a faulty exon is replaced by a correct exon. The present invention can conveniently be used for the treatment of cystic fibrosis, preferably by exchange of aberrant exon 10 of CFTR (cystic fibrosis transmembrane conductance regulator) for a correct version of exon 10 (SEQ ID NO: 1). The present invention can also conveniently be used for the treatment of other diseases or disorders. Accordingly, the present invention can conveniently be used for making a change in a target RNA molecule associated with a disorder and/or the treatment of diseases related to (genetic) disorders, such as but not limited to albinism, alpha-1-antitrypsin deficiency, Alzheimer disease, Amyotrophic lateral sclerosis, Asthma, β-thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidormylosis bullosa, Fabry disease, Familial Adenomatous, Polyposis, Galactosemia, Gaucher's Disease, Glucose-6-phosphate dehydrogenase, Haemophilia, Hereditary Hematochromatosis, Hunter Syndrome, Huntington's disease, Hurler Syndrome, Inflammatory Bowel Disease (IBD), Inherited polyagglutination syndrome, Lesch-Nyhan syndrome, Lynch, Marfan syndrome, Mucopolysaccharidosis, Muscular Dystrophy, Myotonic dystrophy types I and II, Niemann-Pick disease type A, B and C, NY-esol related cancer, Parkinson's disease, Peutz-Jeghers Syndrome, Phenylketonuria, Pompe's disease, Primary Ciliary Disease, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease, X-linked immunodeficiency, various forms of cancer (e.g. BRCA1 and 2 linked breast cancer and ovarian cancer), and the like.

While the invention will primarily be used to repair defects associated with disease or a disorder in a target mRNA, the invention is not limited to such use. As will be readily understood by a person having ordinary skill in the art, any exon sequence may be exchanged by any other exon sequence, for example for purposes of studying the effects of certain mutations in the encoded protein, creation of stop codons, protein engineering and the like. Although the exon carrying the change vis-à-vis the exon in the target RNA is sometimes referred to as “the artificial” exon, it should be understood that this could refer to an exon that is a naturally occurring exon, even the preferred wild-type exon.

The artificial exon is preferably present on a nucleic acid molecule according to the invention, preferably a piece of RNA, which is in vitro generated and stabilized. The nucleic acid molecule according to the invention, preferably a piece of artificial RNA, may be generated by de novo synthesis. Alternatively it may be generated by in vitro transcription. Alternatively it may be generated by in vivo transcription.

In order to increase specific trans-splicing efficiency the nucleic acid molecule according to the invention, preferably an RNA molecule, can base-pair with parts of the introns that surround the exon to-be-replaced. The nucleic acid molecule according to the invention, preferably an artificial RNA, preferably also encodes the branch point (BP) and the polypyrimidine tract, as well as the 3′ and 5′ splice sites bordering the exon. In addition the molecule could contain a spacer sequence between the base pairing region and the neighboring element. In addition the molecule could contain intronic splicing enhancers (ISE) to increase trans-splicing efficiency.

The region for base-pairing can be anywhere within the introns surrounding the exon to-be-replaced. The length for base-pairing is between 50 and 250 nucleotides. The branch point could have the consensus sequence tactaactgt (SEQ ID NO: 2), but since the sequence of the branch points is poorly conserved in mammals alternatives such as ctaat (SEQ ID NO: 3) or others could also be used. The polypyrimidine tract could have the consensus sequence cctttcttcttttccttcc (SEQ ID NO: 4). Alternatively it could have the sequence ttttatttcc (SEQ ID NO: 5) or any other sequence of at least nine thymine or cytosine nucleotides. The 5′ and 3′ splice sites could be the ones naturally surrounding the exon to-be-replaced. Alternatively they can be the consensus sequences gtaagt (SEQ ID NO: 6) and tccctccag (SEQ ID NO: 7) for 5′ and 3′ splice sites, respectively.

The present invention is directed to a method to preferably replace exon 10 of CFTR (cystic fibrosis transmembrane conductance regulator, SEQ ID NO: 1). This can be applied to treat patients with a mutation in exon 10, such as ΔF508.

The RNA used for replacement of CFTR exon 10 could have the sequence as set forth here below (SEQ ID NO; 8) (exon sequence underlined, SEQ ID NO: 1):

uccaauuaucauccuaagcagaaguguauauucuuauuuguaaag
auucuauuaacucauuugauucaaaauauuuaaaauacuuccugu
uucagguacucugcuaugcacaaaagauacaagggaaaguaaaag
agacaggcaagugaauccugagcgugauuugauaaugaccuaaua
augauggguuuuauuuccagacuucacuucuaauggugauuaugg
gagaacuggagccuucagaggguaaaauuaagcacaguggaagaa
uuucauucuguucucaguuuuccuggauuaugccuggcaccauua
aagaaaauaucaucuuugguguuuccuaugaugaauauagauaca
gaagcgucaucaaagcaugccaacuagaagagguaagaaacucuc
uuucuuuccauggguuggccuugauccauucacaguagcuuaccc
auagaggaaacauaaauauauguagacuaaccgauugaauaugga
gccaaauauauaauuuggguagugugaaggguucauaugcauaau
caaaaaguuuucacauaguuucuuac

Alternatively the exon replacement molecule could have the sequence as set forth here below (SEQ ID NO:9) (exon sequence underlined, SEQ ID NO: 1):

uccaauuaucauccuaagcagaaguguauauucuuauuuguaaag
auucuauuaacucauuugauucaaaauauuuaaaauacuuccugu
uucagguacucugcuaugcacaaaagauacaagggaaaguaaaag
agacagauaaugaccuacuaacugugccuuucuucuuuuccuucc
agacuucacuucuaauggugauuaugggagaacuggagccuucag
aggguaaaauuaagcacaguggaagaauuucauucuguucucagu
uuuccuggauuaugccuggcaccauuaaagaaaauaucaucuuug
guguuuccuaugaugaauauagauacagaagcgucaucaaagcau
gccaacuagaagagguaagaaacucucuuucuuuccauggguugg
ccuugauccauucacaguagcuuacccauagaggaaacauaaaua
uauguagacuaaccgauugaauauggagccaaauauauaauuugg
guagugugaaggguucauaugcauaaucaaaaaguuuucacauag
uuucuuac

Alternatively the base-pairing sequences could be derived anywhere from the introns surrounding CFTR exon 10, one example is SEQ ID NO: 10 (intron sequences in bold, exon sequence underlined, SEQ ID NO: 1):

ugccaagugcucacucugugucgagugcuguucuaugugcuuuaa
cuauauuaauuuauuuaaucuucacagaaauccuacaaaguagau
uaccuucauauuauuagguacagauuaaguaauagagacauauuc
agguagauaaugaccuacuaacugugccuuucuucuuuuccuucc
agacuucacuucuaauggugauuaugggagaacuggagccuucag
aggguaaaauuaagcacaguggaagaauuucauucuguucucagu
uuuccuggauuaugccuggcaccauuaaagaaaauaucaucuuug
guguuuccuaugaugaauauagauacagaagcgucaucaaagcau
gccaacuagaagagguaagaaacucucuuucuuuccauggguugg
ccuaaauaaucuuaauaauuuuuggaguauauuuuuaaagaugca
uauuuugugguaucuuuuaaaaagauaccacauaucacuuauaug
caugccauauaaauaaccauugaggacguuugucucacuaaugag
ugaacaaa

The nucleic acid molecule according to the invention, preferably an artificial RNA, is preferably stabilized to improve its survival in the body and in cells. Alterations to improve stabilization could be 2′-O-Me or 2′Fluo modified RNA nucleotides. Alternatively, 2-thiouridine and/or 5-methyl-cytidine could be applied. These could be introduced during a chemical or natural polymerization reaction. Alternatively LNAs could be inserted. Alternatively nucleotides could for example be coupled using phosphorothioate or methylphosphonate linkages to increase stability. For application in vivo the exon replacement nucleic acid molecules according to the invention, preferably RNA molecules, might be delivered in a liposome, polysome, or nanoparticle. Alternatively the exon exchange molecules might be complexed to polyethylene-imine (PEI) and/or polyethylene glycol (PEG), or linked to a sterol, preferably cholesterol, or any other commercially available compound intended for RNA delivery.

Many medicines intended for the lung can be applied via the airway. One such a medicine could be the nucleic acid molecule according to the invention, preferably a stabilized RNA, intended for exon replacement. In many diseases the mucus layer shows an increased thickness, leading to a decreased absorption of medicines via the lung. One such a disease is chronical bronchitis, another example is cystic fibrosis. Various forms of mucus normalizers are available, such as a DNAse, mannitol, or a small molecule for treatment of CF, preferably Kalydeco (ivacaftor; VXVX-770), VX-809 (Lumacaftor) and/or VX-661. When mucus normalizers are used in combination with exon replacement RNA compounds they can increase the effectivity of those medicines. Therefore the combination of a mucus normalizer with an exon replacer molecule, potentially in a delivery particle, might increase functionality the exon replacement.

Nucleic acid molecules according to the invention are typically administered in doses ranging from 1 μg to 1000 mg, more preferably from 10 μg to 100 mg, still more preferable from 100 μg to 10 mg, and most preferably 500 μg to 5 mg depending on the cell (tissue) to be treated, the weight of the organism, the mode and/or site of administration (local vs. systemic, the site of administration (intraperitoneal, intramuscular, pulmonary, etc.), the disorder to be treated, the regimen to be applied (single or repeated bolus or continuous dosing) and the like. A person having ordinary skill in the art will be capable of establishing the optimal dose using some trial and error.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 0.1% of the value.

The sequence information as provided herein should not be so narrowly construed as to require inclusion of erroneously identified nucleotides. The skilled person is capable of identifying such erroneously identified nucleotides and knows how to correct for such errors. In case of sequence errors, the genomic DNA, mRNA and polynucleotide sequences of the cystic fibrosis transmembrane conductance regulator (CFTR) should prevail.

All patent and literature references cited in the present specification are hereby incorporated by reference in their entirety.

### FIGURE LEGENDS

FIG. 1. Schematic drawing of a exon replacement molecule. The intronic splicing enhancers are optional. A spacer sequence might be present between the base pairing regions an the neighboring elements. ISE, intronic splicing enhancers; polypyr., polypyrimidine tract; ss, splice site.

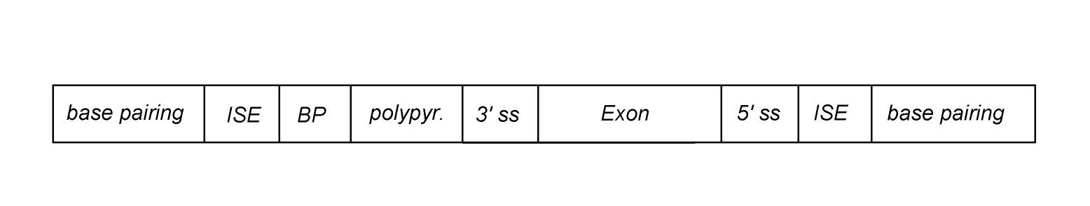

1. Use of a nucleic acid molecule for the treatment or prevention of a disease related to a genetic disorder in a subject, preferably a human subject, comprising administration of the nucleic acid molecule to the subject, wherein said nucleic acid molecule comprises:
a. a first polynucleotide to be trans-spliced to a pre-mRNA, said first polynucleotide encoding at least part of the amino acid sequence encoded by the wild-type pre-mRNA, or of at least part of an amino acid sequence that has at least 95% sequence identity to the amino acid sequence encoded by the wild-type pre-mRNA;
b. a second polynucleotide flanking the first polynucleotide on the 5′ side, comprising from 5′ to 3′: at least a sequence in reverse complement to a sequence of the pre-mRNA flanking the sequence to be trans-spliced from the pre-mRNA on the 5′, a branch point, a polypyrimidine tract and a 3′splice acceptor, and optionally comprising at least one of: an intronic splice enhancer and a spacer between the reverse complement sequence and the branch point;
c. a third polynucleotide flanking the first polynucleotide on the 3′ side, comprising from 5′ to 3′: at least a 5′ splice donor site and a sequence in reverse complement to a sequence of the pre-mRNA flanking the sequence to be trans-spliced from the pre-mRNA on the 3′, and optionally comprising at least one of: an intronic splice enhancer and a spacer between the 5′ splice donor site and the reverse complement sequence.

2. Use according to claim 1, wherein the reverse complement sequences have a length of 50-250 nucleotides, preferably 70-200 nucleotides, more preferably 70-150 nucleotides.

3. Use according to claim 1 or 2, wherein the reverse complement sequences are in reverse complement to intron sequences flanking the sequence to be trans-spliced from the pre-mRNA.

4. Use according to any of the preceding claims wherein the sequence to be trans-spliced is an exon.

5. Use according to any of the preceding claims, wherein the nucleic acid molecule is stabilized by comprising modified nucleotides, preferably selected from the group consisting of a 2′-0 methyl ribose, 2′Fluoro ribose, phosphorotioate, methylphosphonate, PMO, 5-methyl-dC, 2-amino-dA, C5-pyrimidine, 2-thiouridine and/or 5-methyl-cytidine.

6. Use according to any of the preceding claims, wherein the nucleic acid molecule comprises RNA, DNA, PNA and/or LNA.

7. Use according to any of the preceding claims, wherein the branch point has the consensus sequence tactaactgt (SEQ ID NO: 2) or ctaat (SEQ ID NO: 3).

8. Use according to any of the preceding claims, wherein the polypyrimidine tract has the consensus sequence cctttcttcttttccttcc (SEQ ID NO: 4) or ttttatttcc (SEQ ID NO: 5) or comprises at least nine pyrimidines.

9. Use according to any of the preceding claims, wherein the 5′ splice donor has the consensus sequence gtaagt (SEQ ID NO: 6) and/or 3′ splice acceptor has the consensus sequence tccctccag (SEQ ID NO: 7).

10. Use according to any of the preceding claims, wherein the disease related to a genetic disorder is cystic fibrosis and the genetic disorder is an aberrant exon 10 of the CFTR gene.

11. Use according to any of the preceding claims, wherein the sequence to be trans-spliced is at least exon 10 of the CFTR gene.

12. Use according to any of the preceding claims, wherein:
a. the second polynucleotide comprising at least 50 nucleotides having at least 75%, 80%, 85%, 90%, 95%, or 99% sequence identity to nucleotides 1-166 of SEQ ID NO: 8;
b. the first polynucleotide encoding the amino acid sequence encoded by nucleotides 200-392 of SEQ ID NO: 8, or encoding an amino acid sequence that has at least 95% sequence identity to the amino acid sequence encoded by nucleotides 200-392 of SEQ ID NO: 8;
c. the third polynucleotide comprising at least 50 nucleotides having at least 75%, 80%, 85%, 90%, 95%, or 99% sequence identity to nucleotides 427-566 of SEQ ID NO: 8.

13. Use according to any of the preceding claims, wherein:
a. the second polynucleotide comprises at least 50 nucleotides having at least 75%, 80%, 85%, 90%, 95%, or 99% sequence identity to nucleotides 1-140 of SEQ ID NO: 9;
b. the first polynucleotide encodes the amino acid sequence encoded by nucleotides 182-374 of SEQ ID NO: 9, or encoding an amino acid sequence that has at least 95% sequence identity to the amino acid sequence encoded by nucleotides 182-374 of SEQ ID NO: 9;
c. the third polynucleotide comprises at least 50 nucleotides having at least 75%, 80%, 85%, 90%, 95%, or 99% sequence identity to nucleotides 408-548 of SEQ ID NO: 9.

14. Use according to any of the preceding claims, wherein:
a. the second polynucleotide comprises at least 50 nucleotides having at least 75%, 80%, 85%, 90%, 95%, or 99% sequence identity to nucleotides 1-140 of SEQ ID NO: 10;
b. the first polynucleotide encodes the amino acid sequence encoded by nucleotides 182-374 of SEQ ID NO: 10, or encoding an amino acid sequence that has at least 95% sequence identity to the amino acid sequence encoded by nucleotides 182-374 of SEQ ID NO: 10;
c. the third polynucleotide comprises at least 50 nucleotides having at least 75%, 80%, 85%, 90%, 95%, or 99% sequence identity to nucleotides 408-548 of SEQ ID NO: 10.

15. Use according to any of the preceding claims, wherein the nucleic acid molecule consists of SEQ ID NO: 8, SEQ ID NO: 9 or SEQ ID NO: 10.

16. Use according to any of the preceding claims, wherein the nucleic acid molecule is administered in a vehicle, preferably a liposome, polysome, or nanoparticle and/or wherein the nucleic acid molecule is complexed to a delivery compound, preferably polyethylene-imine (PEI), polyethyleneglycol (PEG), or linked to a sterol, preferably cholesterol.

17. Use according to any of the preceding claims, wherein the nucleic acid molecule is administered to the lung, preferably via the airways, and preferably the nucleic acid molecule is administered together with a transfection mediator.

18. A nucleic acid molecule as defined in any of claims 1-17.

19. A pharmaceutical composition comprising a nucleic acid molecule according to claim 18 and a pharmaceutical acceptable carrier.

20. A pharmaceutical composition according to claim 19, further comprising a transfection mediator.

21. A pharmaceutical composition according to claim 19 or 20 further comprising a cystic fibrosis medicine known to the person skilled in the art, preferably a DNase and/or mannitol and/or a small molecule for treatment of CF, preferably Kalydeco (ivacaftor; VXVX-770), VX-809 (Lumacaftor) and/or VX-661.

22. A nucleic acid molecule according to claim 18 or a composition according to any one of claims 19-21 for use in the treatment or prevention of cystic fibrosis.

23. A method for the prevention or treatment of a disease related to a genetic disorder in a subject, preferably a human subject, comprising the administration of a nucleic acid molecule according to claim 18 or a composition according to any one of claims 19-21 to said subject.

24. A method according to claim 23, wherein the disease related to a genetic disorder is cystic fibrosis and the genetic disorder is an aberrant exon 10 of the CFTR gene.

25. A method according to claim 24, wherein the sequence to be trans-spliced is at least exon 10 of the CFTR gene.

26. A method according to any one of claims 23-25, wherein the nucleic acid molecule is administered in a vehicle, preferably a liposome, polysome, or nanoparticle and/or wherein the nucleic acid molecule is complexed to a delivery compound, preferably polyethylene-imine (PEI), polyethyleneglycol (PEG), or linked to a sterol, preferably cholesterol.

27. A method according to any one of claims 23-26, wherein the nucleic acid molecule is administered to the lung, preferably via the airways, and preferably the nucleic acid molecule is administered together with a transfection mediator.

28. An in vitro or in vivo method of exon replacement by trans-splicing, comprising contacting a pre-mRNA with a nucleic acid molecule according to claim 18, a composition comprising a nucleic acid molecule according to claim 18, or a composition according to any one of claims 19-21.

29. A method according to claim 28, wherein the exon to be trans-spliced is at least exon 10 of the CFTR gene.

30. A nucleic acid molecule for use in the treatment or prevention of a disease related to a genetic disorder in a subject, preferably a human subject, comprising administration of the nucleic acid molecule to the subject, wherein said nucleic acid molecule comprises:
a. a first polynucleotide to be trans-spliced to a pre-mRNA, said first polynucleotide encoding at least part of the amino acid sequence encoded by the wild-type pre-mRNA, or of at least part of an amino acid sequence that has at least 95% sequence identity to the amino acid sequence encoded by the wild-type pre-mRNA;
b. a second polynucleotide flanking the first polynucleotide on the 5′ side, comprising from 5′ to 3′: at least a sequence in reverse complement to a sequence of the pre-mRNA flanking the sequence to be trans-spliced from the pre-mRNA on the 5′, a branch point, a polypyrimidine tract and a 3′ splice acceptor site, and optionally comprising at least one of: an intronic splice enhancer and a spacer between the 3′ splice acceptor site and the branch point;
c. a third polynucleotide flanking the first polynucleotide on the 3′ side, comprising from 5′ to 3′: at least a 5′ splice donor site and a sequence in reverse complement to a sequence of the pre-mRNA flanking the sequence to be trans-spliced from the pre-mRNA on the 3′, and optionally comprising at least one of: an intronic splice enhancer and a spacer between the 5′ splice donor site and the reverse complement sequence.

31. A nucleic acid molecule according to claim 30, wherein the reverse complement sequences have a length of 50-250 nucleotides, preferably 70-200 nucleotides, more preferably 70-150 nucleotides.

32. A nucleic acid molecule according to claim 30 or 31, wherein the reverse complement sequences are in reverse complement to intron sequences flanking the sequence to be trans-spliced from the pre-mRNA.

33. A nucleic acid molecule according to any of claims 30-32 wherein the sequence to be trans-spliced is an exon.

34. A nucleic acid molecule according to any of claims 30-33, wherein the nucleic acid molecule is stabilized by comprising modified nucleotides, preferably selected from the group consisting of a 2′-0 methyl ribose, 2′Fluoro ribose, phosphorotioate, methylphosphonate, PMO, 5-methyl-dC, 2-amino-dA, C5-pyrimidine, 2-thiouridine and/or 5-methyl-cytidine.

35. A nucleic acid molecule according to any of claims 30-34, wherein the nucleic acid molecule comprises RNA, DNA, PNA and/or LNA.

36. A nucleic acid molecule according to any of claims 30-35, wherein the branch point has the consensus sequence tactaactgt (SEQ ID NO: 2) or ctaat (SEQ ID NO: 3).

37. A nucleic acid molecule according to any of claims 30-36, wherein the polypyrimidine tract has the consensus sequence cctttcttcttttccttcc (SEQ ID NO: 4) or ttttatttcc (SEQ ID NO: 5) or comprises at least nine pyrimidines.

38. A nucleic acid molecule according to any of claims 30-37, wherein the 5′ splice donor has the consensus sequence gtaagt (SEQ ID NO: 6) and/or 3′ splice acceptor has the consensus sequence tccctccag (SEQ ID NO: 7).

39. A nucleic acid molecule according to any of claims 30-38, wherein the disease related to a genetic disorder is cystic fibrosis and the genetic disorder is an aberrant exon 10 of the CFTR gene.

40. A nucleic acid molecule according to claim of claims 30-39, wherein the sequence to be trans-spliced is at least exon 10 of the CFTR gene.

41. A nucleic acid molecule according to any of claims 30-40, wherein:
a. the second polynucleotide comprises at least 50 nucleotides having at least 75%, 80%, 85%, 90%, 95%, or 99% sequence identity to nucleotides 1-166 of SEQ ID NO: 8;
b. the first polynucleotide encodes the amino acid sequence encoded by nucleotides 200-392 of SEQ ID NO: 8, or encoding an amino acid sequence that has at least 95% sequence identity to the amino acid sequence encoded by nucleotides 200-392 of SEQ ID NO: 8;
c. the third polynucleotide comprises at least 50 nucleotides having at least 75%, 80%, 85%, 90%, 95%, or 99% sequence identity to nucleotides 427-566 of SEQ ID NO: 8.

42. A nucleic acid molecule according to any of claims 30-41, wherein:
a. the second polynucleotide comprises at least 50 nucleotides having at least 75%, 80%, 85%, 90%, 95%, or 99% sequence identity to nucleotides 1-140 of SEQ ID NO: 9;
b. the first polynucleotide encodes the amino acid sequence encoded by nucleotides 182-374 of SEQ ID NO: 9, or encoding an amino acid sequence that has at least 95% sequence identity to the amino acid sequence encoded by nucleotides 182-374 of SEQ ID NO: 9;
c. the third polynucleotide comprises at least 50 nucleotides having at least 75%, 80%, 85%, 90%, 95%, or 99% sequence identity to nucleotides 408-548 of SEQ ID NO: 9.

43. A nucleic acid molecule according to any of claims 30-42, wherein:
a. the second polynucleotide comprises at least 50 nucleotides having at least 75%, 80%, 85%, 90%, 95%, or 99% sequence identity to nucleotides 1-140 of SEQ ID NO: 10;
b. the first polynucleotide encodes the amino acid sequence encoded by nucleotides 182-374 of SEQ ID NO: 10, or encoding an amino acid sequence that has at least 95% sequence identity to the amino acid sequence encoded by nucleotides 182-374 of SEQ ID NO: 10;
c. the polynucleotide comprises at least 50 nucleotides having at least 75%, 80%, 85%, 90%, 95%, or 99% sequence identity to nucleotides 408-548 of SEQ ID NO: 10.

44. A nucleic acid molecule according to any of claims 30-43, wherein the nucleic acid molecule consists of SEQ ID NO: 8, SEQ ID NO: 9 or SEQ ID NO: 10.

45. A nucleic acid molecule according to any of claims 30-44, wherein the nucleic acid molecule is administered in a vehicle, preferably a liposome, polysome, or nanoparticle and/or wherein the nucleic acid molecule is complexed to a delivery compound, preferably polyethylene-imine (PEI), polyethyleneglycol (PEG), or linked to a sterol, preferably cholesterol.

46. A nucleic acid molecule according to any of claims 30-45, wherein the nucleic acid molecule is administered to the lung, preferably via the airways, and preferably the nucleic acid molecule is administered together with a transfection mediator and/or a cystic fibrosis medicine known to the person skilled in the art, preferably a DNase, mannitol and/or a small molecule for treatment of CF, preferably Kalydeco (ivacaftor; VXVX-770), VX-809 (Lumacaftor) and/or VX-661.
